(19) United States
(12) Patent Application Publication
(10) Pub. No.: US 2023/0021568 A1
(43) Pub. Date: Jan. 26, 2023

(54) METHOD AND SYSTEM TO PREDICT PROGNOSIS FOR CRITICALLY ILL PATIENTS

(71) Applicant: CARESTREAM HEALTH, INC., Rochester, NY (US)

(72) Inventor: David H. FOOS, Webster, NY (US)

(21) Appl. No.: 17/791,041

(22) PCT Filed: Nov. 19, 2020
(86) PCT No.: PCT/US2020/061152

§ 371 Date: Jul. 6, 2022

Related U.S. Application Data
(60) Provisional application No. 62/959,211, filed on Jan. 10, 2020.

(51) Int. Cl. G16H 50/20; A61B 6/00; A61B 6/02; G16H 30/40

(52) U.S. Cl. CPC G16H 50/20; A61B 6/4405; A61B 6/025; A61B 6/5217; A61B 6/482; G16H 30/40; A61B 6/5205

(57) ABSTRACT

A method for evaluating one or more diagnostic linages of a patient obtained in different examination sessions and evaluating the diagnostic images using trained machine learning logic to generate prognosis and treatment information related to a medical condition of the patient detected during the evaluation. The prognosis-related information is recorded and displayed.

### TECHNICAL FIELD

The disclosure relates generally to the field of patient assessment and treatment and more particularly to tracking and use of patient data acquired for a patient in an intensive care unit (ICU) or related facility for generating prognosis and treatment information based on the acquired data.

### BACKGROUND

Monitoring health status, including respiratory status, is a critically important aspect of ongoing care for patients who are admitted to a hospital intensive care unit (ICU). Continuous or periodic monitoring of a patient health status forms the basis for healthcare providers to make adjustments to the patient's treatment regimen.

Methods that are used for monitoring ICU patient health status include using devices for tracking vital signs, such as to measure heart rate, blood pressure, blood oxygen level, and other patient parameters. Many of these devices perform continuous monitoring, however, some devices are used intermittently, such as portable chest X-ray, which can be used daily or may be used multiple times daily to assess changes in respiratory condition.

Considerable amounts of measured data, from instruments as well as from images, are collected during a typical patient stay in the ICU. Particular elements of this data can be meaningful to various specialists and can guide their treatment recommendations for specific conditions. However, considering the totality of the information that is typically obtained, it can be difficult for the attending staff to take all of the relevant patient data into consideration for directing or modifying the treatment regimen for a patient, particularly under the demanding time, scheduling, and budgetary constraints of the ICU environment.

In today's standard of care, change assessment is derived from longitudinal monitoring of a patient's health status and thus forms the basis for caregivers to specify and to make refinements to treatment protocols. As such, conventional ICU treatment refinement is an intrinsically reactive process, with treatment practices primarily driven in short-term response to the most recent changes in measurements and their relative urgency. A thorough, detailed analytical assessment of the patient's condition and prognosis can be difficult to obtain in the ICU environment. In terms of staff time and effort, short-term concerns readily take precedence over longer-term considerations or projection of patient outcome. Thus, as the patient condition progresses or recedes, for instance, such as with fluid levels increasing or decreasing in the lungs, or various vital signs changing at a rapid rate, the treatment regimen is largely determined based on the most recent changes in the patient condition, in response to the current treatment practices.

By comparison with patient data obtained from typical vital signs measurements, image data can have significantly more information content, particularly when images acquired in sequence over a period of time are compared against each other, such as to show disease development or rate of change of a particular life-threatening condition, for example. At the same time, image content can be challenging to accurately interpret, particularly for staff handling the demands of an urgent care environment. Thus, subtle changes in patient condition may be detectable in a progressive series of images taken in the ICU, but may not be accurately detected where attention is given only to the latest available data.

It can be appreciated that there would be significant benefit in tools that can assist the attending practitioner in analyzing patient condition and that can help to generate a prognosis and provide guidance for formulating or altering a particular patient regimen according to image content and supported by the vast body of patient data that is available from instrumentation, vital signs measurement, and patient history.

### SUMMARY

Objects of the present disclosure include advancing the value of radiographic imaging for the broader purpose of overall patient assessment and treatment and addressing the shortcomings relative to prognosis development noted previously in the background section. With these and related objects in mind, embodiments described herein address the need for making more effective use of imaging and measurement data related to patient condition, particularly for the patient in an ICU setting.

These objects are given only by way of illustrative example, and such objects may be exemplary of one or more embodiments of the invention. Other desirable objectives and advantages inherently achieved may occur or become apparent to those skilled in the art. The invention is defined by the appended claims.

According to one aspect of the disclosure, there is provided a method for evaluating diagnostic images of a patient, the method comprising acquiring diagnostic images of the patient during different examination sessions and evaluating the diagnostic images using trained machine learning logic to generate prognosis and treatment information for the patient applicable to a medical condition of the patient that is detected during the evaluation. The prognosis and treatment information may be output, recorded, displayed, printed, or a combination thereof.

### BRIEF DESCRIPTION OF THE DRAWINGS

The foregoing and other objects, features, and advantages of the invention will be apparent from the following more particular description of the embodiments of the invention, as illustrated in the accompanying drawings. The elements of the drawings are not necessarily to scale relative to each other.

FIG. 1A is a schematic diagram that shows interaction between patient data acquisition and analysis tools for supporting prognosis generation according to an embodiment of the present disclosure.

FIG. 1B is a schematic diagram that shows an alternative arrangement, in which updated patient history is extracted from the overall patient records.

FIG. 2 shows an exemplary portable diagnostic imaging unit for bedside radiography.

FIG. 3 is a logic flow diagram that shows a sequence for image processing that can take advantage of machine learning software and patient history, including use of image content obtained previously.

FIGS. 4A and 4B show exemplary interface display arrangements that can provide useful reporting of prognosis data and projected treatment strategies.

FIG. 5 is a plan view that shows a tomosynthesis reconstruction using a set of projection images within a narrow angular range for a phantom chest image, identifying particular areas of interest that have been detected using learned logic.

### DETAILED DESCRIPTION OF THE EMBODIMENTS

This application claims the benefit of U.S. Provisional application U.S. Ser. No. 62/959,211, provisionally filed on Jan. 10, 2020, entitled “METHOD AND SYSTEM TO PREDICT PROGNOSIS FOR CRITICALLY ILL PATIENTS”, in the name of David H. Foos, hereby incorporated by reference herein in its entirety.

The following is a detailed description of the preferred embodiments, reference being made to the drawings in which the same reference numerals identify the same elements of structure in each of the several figures.

Where they are used, the terms “first”, “second”, and so on, do not necessarily denote any ordinal or priority relation, but may be used for more clearly distinguishing one element or time interval from another. The term “plurality” means at least two.

In the context of the present disclosure, the terms “viewer”, “operator”, and “user” are considered to be equivalent and refer to the viewing practitioner or other person who views and manipulates equipment for x-ray acquisition or an x-ray image itself on a display monitor. An “operator instruction” or “viewer instruction” is obtained from explicit commands entered by the viewer using an input device, such as a computer mouse or keyboard.

The term “in signal communication” as used in the application means that two or more devices and/or components are capable of digitally communicating with each other via signals that travel over some type of signal path. Signal communication may be wired or wireless. The signals may be communication, power, data, or energy signals which may communicate information, power, and/or energy from a first device and/or component to a second device and/or component along a signal path between the first device and/or component and second device and/or component. Signal paths may include physical, electrical, magnetic, electromagnetic, optical, wired, and/or wireless connections between the first device and/or component and second device and/or component. Signal paths may also include additional devices and/or components between the first device and/or component and second device and/or component.

In the context of the present disclosure, the term “intensive care unit” or ICU has its conventional meaning, describing an environment in which patients considered to be dangerously ill are treated under constant observation. While embodiments of the present disclosure address some of the particular needs characteristic of the ICU environment, the apparatus and methods described herein are not limited to ICU facilities, but can be applicable to emergency room patients as well as a more general hospital patient population.

For execution and orchestration of the various tasks and capabilities listed hereinabove, embodiments of the present invention can employ trained logic or learned logic, equivalently termed “machine learning”, and utilize various tools and capabilities. Trained or machine-learned logic can be distinguished from conventional programmed logic that is formulated based on a formal instruction language that is used by a programmer to specify particular data operations to be performed by a processor or processing system. In various embodiments, the processing system can include portions of executable code that have been generated using conventional procedural programming that provides a predictable response according to received inputs, as well as other portions of executable code that have been generated using machine learning techniques that are characterized as model-based and probabilistic, based on training using multiple examples, and provide solutions derived from heuristic processes.

Measured data from the patient can include instrument data from various types of monitoring devices having sensors that obtain vital signs data.

Flat panel digital radiography (DR) is widely deployed across many geographic areas. DR has proven to be safe and effective for many clinical indications such as for suspected fractures, shortness of breath, in emergency departments to image patients following motor vehicle accidents or other trauma circumstances, and for bedside imaging in the critical care (intensive care unit) setting, to monitor patient respiratory status, for example.

Among potential benefits, embodiments of the present disclosure address the need to make more effective use of the totality of patient data that can typically be obtained for the critically ill patient in the ICU, with particular focus on changes to imaging and measurement data acquired during the patient's stay in the ICU. In an embodiment of the present invention, machine learning can be used to help guide treatment as well as to predict a patient's prognostic outlook, near-term or over a longer interval. Treatment guidance can be provided by a schedule displayed by a patient tracking system that employs learned logic, for example.

The learned logic, which can be considered the machine learning algorithm output, can be implemented using a trained neural network or using a statistical model-based method that utilizes data that has been collected about a patient during an ICU stay. This data may include, but is not limited to, imagery obtained in one or more examination sessions using portable chest x-ray, tomosynthesis, computed tomography, dual energy x-ray, x-ray motion sequences obtained using serial radiography, ultrasound imagery, patient demographic information, clinical history, histologic, genomic and proteomic information, and measurements taken regarding the patient's vital signs. The machine learning process can be trained to predict prognosis by leveraging instances of the aforementioned data obtained from prior patients in numerous test cases, for which patient outcomes are also known and have been carefully documented.

The learned logic output, i.e., the prognostic outlook that is reported for the patient, can take many forms. According to an embodiment of the present disclosure, the output from machine learning can be in the form of probabilistic metrics or indicators. For example, these indicators could include metrics on probability of mortality as a function of time and probability of disease progression as a function of time. Additional machine learning outputs may include recommendations or suggestions for changes in treatment regimen that may increase the probability (and timing) for a positive patient outcome. For example, changes in treatment regimen could include adjustments to respirator settings, modification to flow rates for IV fluid, and changes to antibiotic concentration, among others.

The analysis executed herein using machine learning is conventionally performed by practitioners who may specialize in fields of cardiology, pathology, radiology, and other fields. As noted previously, the volume of patient data that is obtained in the ICU can make it difficult for any individual practitioner to consider more than a portion of the data that is specifically related to one or another discipline. Image data, such as images obtained using bedside DR apparatus, can be particularly useful, but may be difficult to interpret properly without significant training and practice.

Machine learning provides an opportunity to benefit from the multifaceted information that is obtained and to make decisions informed by analysis of the broad base of available data. This capability does not replace the practitioner, but can provide the benefits of diagnostic assistance using machine learning that is based on probabilistic analysis of historical data for thousands of patients. Machine learning is particularly advantaged for its ability to derive useful information from complex patterns of data. In the case of an ICU patient, the large body of data that machine learning can handle can exceed that available or of immediate interest to the specialist. It can be appreciated that, in many cases, full information available about the patient can include a broader range of metrics, imaging content, and historical or genetic data than might normally be reviewed or analyzed by, or normally be requested by any single specialist. Much of the available data related to the patient can lie outside the body of data normally reviewed by the practitioner in addressing the condition of the ICU patient.

An embodiment of the present disclosure can use a modular approach to patient data assessment, suited to the demands of the ongoing and periodic test, bedside imaging, and vital signs measurements that are typical of the ICU environment. Following this model, different processing modules can be used to assess the various types of data that are obtained, capable of providing useful information for supporting short-term remedial activities of the ICU staff. Thus, for example, sudden changes in patient condition or measurements, or combinations of measurements, outside of desirable range, can be reported as more urgent data requiring short-term response. This same data can also be useful for longer-term prognosis considerations for the patient. Thus, results of more specialized processing from any module can be directed to processing logic that is trained for a more holistic approach and that supports longer-term treatment regimen and prognosis generation.

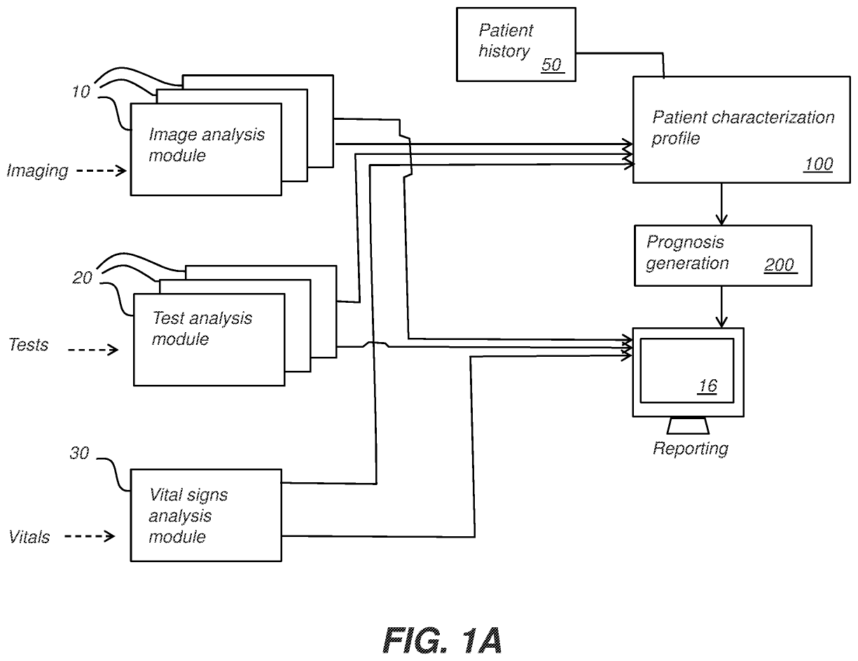

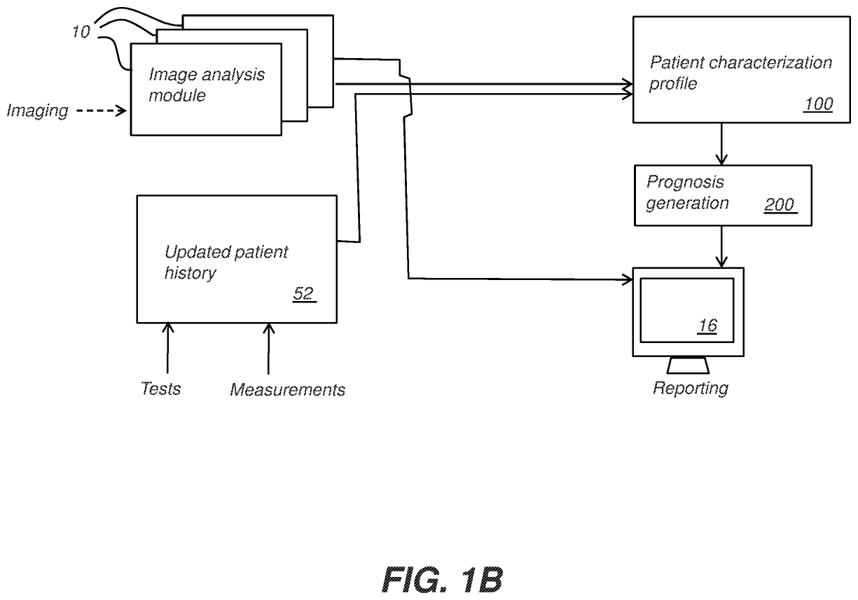

The modular organization given schematically in FIG. 1A shows a set of more specialized modules developed to process and provide some response for specific types of patient data, as well as to provide input to a patient characterization profile 100.

An image analysis module 10 accepts acquired images obtained in an examination session as input and performs the needed image analysis for identifying patient condition, reporting results by signals sent to a display 16. Image analysis module 10 also directs image data and any initial analysis information to patient characterization profile 100 for longer-term assessment and consideration with respect to other patient data. As shown in the FIG. 1A schematic, there can be any number of image analysis modules 10, each designed and trained to handle a different type of image and subject anatomy. The type of image content obtained can include radiography as well as other types of image modality. For example, there can be a module 10 that is used for processing AP chest radiography and a separate image analysis module 10 adapted for processing abdominal ultrasound images.

A number of test analysis modules 20 can also be useful for prognosis generation processing, each module configured and trained to evaluate patient data for a particular test type. Test data can include information on presence of infection, as well as information for various results from blood or urine analysis, and other metrics. Test data can be used to automatically update the patient characterization profile 100. A vital signs analysis module 30 can be configured and trained to assess bedside measurements obtained during periodic rounds of technicians and nursing personnel. Vital signs analysis module 30 can help to assess whether or not there is urgency related to any particular measured value or change in value, such as an out-of-range measurement or abrupt change in patient blood pressure or temperature, for example, and provide an alarm or other report message or signal where remedial activity should be initiated. The measured values are also directed to update the patient characterization profile 100.

Patient history 50 can also be combined with results from other test and analysis modules to support generation of characterization profile 100.

Prognosis generation process 200 can work in conjunction with any number of analysis tools that generate and update patient characterization profile 100. Prognosis generation process 200 can provide different types of output, including outputting displayable data such as on a printer or on a display screen, stored reporting and analysis data, and can act as input to treatment scheduling or for alerting staff to patient condition. Prognosis processing can be supplemented by current information relative to health-related environmental factors, as well as regional or local disease or infection data, such as related to an epidemic outbreak or parasite-carried infection, for example.

The system schematic diagram of FIG. 1B shows an alternative arrangement, in which updated patient history 52 is extracted from the overall patient records maintained by the facility or by the patient's health care provider. This simplified model adds image analysis modules 10 and prognosis generation process 200 to the standard medical data records maintained and updated for the patient. In this arrangement, patient characterization profile 100 also serves as a vehicle for organizing the patient data obtained and constantly updated from different sources so that it can be used in combination with image data for generating prognosis data and helping to generate or direct treatment planning.

### Utilizing Image Content

Image analysis modules 10 can be used for processing diagnostic image content from exams obtained from any of a number of types of systems, including both systems that acquire 2D as well as 3D image data. Some exemplary systems that can provide imaging data include digital radiography systems, ultrasound systems, MMR systems, ultrasound apparatus, tomosynthesis devices, computed tomography (CT) systems, cone-beam computed tomography (CBCT) systems, and the like.

Radiographic image analysis is a standard step in diagnosis and radiographic exam sessions may be repeated at regular periods for the ICU patient. For example, chest x-rays may be performed at regular intervals on some ICU patients, particularly where there is likelihood of fluid build-up, shortness of breath, or other problem.

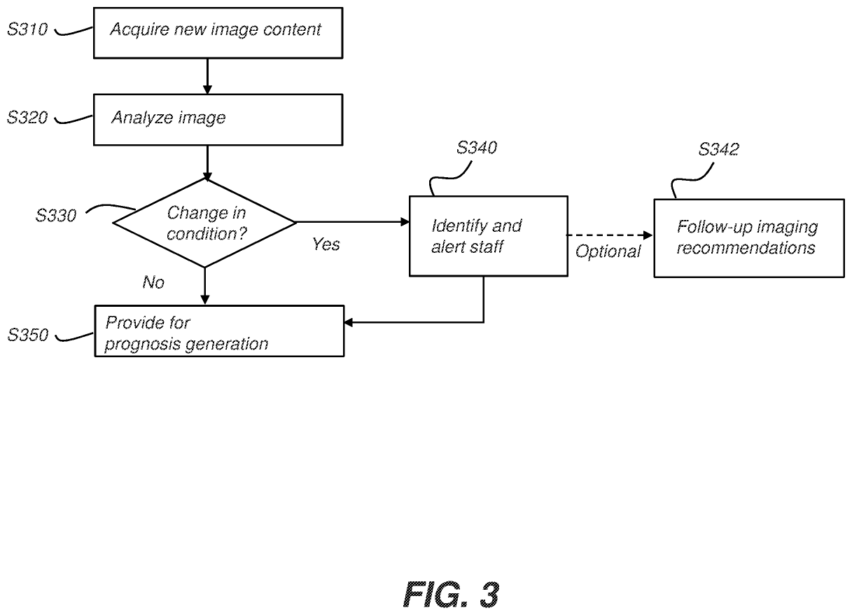

As noted previously, changes in image content can be difficult to evaluate and there can be some delay in obtaining an accurate assessment of image content from the radiography staff. Thus, it can be useful to obtain an initial automated assessment that can suggest a problem that should take priority for response or for advancement to more detailed analysis. The logic flow diagram of FIG. 3 shows a sequence for image processing that can take advantage of machine learning software and patient history, including consideration of image content obtained previously, both while in the ICU and earlier. The new image data is obtained in an image acquisition step S310. An image analysis step S320 can use machine learning or conventional image analysis software to perform a preliminary analysis of image content. Step S320 can reveal, for example, any type of condition that would cause concern, such as excessive lung fluid or other urgent problem requiring more immediate staff attention.

A change assessment step S330 can be configured to compare the most recent image content against previous imaging results and to determine the nature and severity of the change and its implications for patient condition, both near and longer term. Machine learning or learned logic capabilities can be particularly helpful for use as part of change assessment step S330, tracking and interpreting subtle changes in image content over time, including changes that might not be readily detectable to the human observer.

Where change assessment step S330 detects a problem related to transitions in image data, a notification step S340 can execute, reporting change findings and implications to the ICU staff. The image analysis process then executes an image data transmission step S350, providing the image content for subsequent processing in prognosis generation.

An optional follow-up step S342 can be executed based on results and action described with reference to steps S320 and S330. Follow-up step S342 can be guided by learned logic to suggest a follow-up image based on current results. Thus, for example, where a transition indicates a change in patient status, such as significant advancement of infection, malignancy, or illness, or following execution of a treatment or pain alleviation procedure, the learned logic may detect a situation warranting advancement of a normal schedule, such as acquiring a particular image or acquiring standard images at a faster rate than is usual. The imaging apparatus, such as apparatus 110, can further be programmed with learned logic for scheduling recapture of image content over a portion of the anatomy. The processing logic can generate a listing of one or more digital radiography images for subsequent capture, based on analysis of obtained images and other metrics. Messages from the apparatus 110 console can periodically remind the staff of the perceived need for additional diagnostic imaging with any particular patient.

In addition, changes in patient condition that are not directly determined from image content can also serve as input to follow-up step S342. Thus, for example, a dramatic change in body chemistry or a measurement outside of normal or expected values, such as a low oxygen level, may prompt the learned logic to suggest a bedside chest x-ray in an upcoming examination session.

According to an embodiment of the present disclosure, learned logic can also help to direct practitioner attention to locations or regions of interest in an acquired image, as described in more detail subsequently.

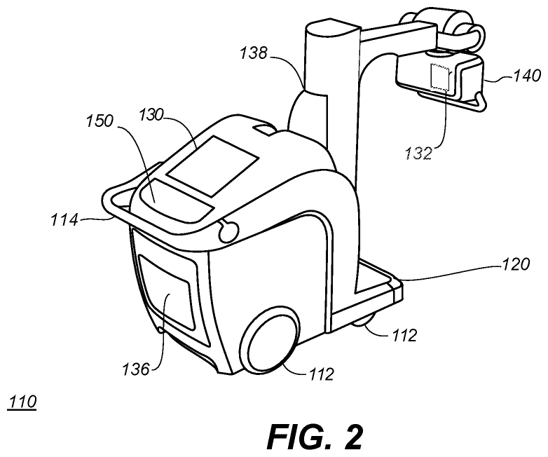

FIG. 2 is a perspective view that shows an exemplary wheeled portable diagnostic imaging unit, mobile radiography apparatus 110 for bedside radiography in an ICU environment. Mobile radiography apparatus 110 can use one or more portable DR detectors adapted to acquire digital image data according to radiation received from the x-ray sources. The exemplary mobile x-ray or radiographic apparatus 110 of FIG. 2 can be employed for digital radiography (DR), pulsed radiography or fluoroscopy, and/or tomosynthesis. As shown in FIG. 2, mobile radiography apparatus 110 can include a moveable transport frame 120 that includes a first display 130 and an optional second display 132 to display relevant information such as obtained images and related data. As shown in FIG. 2, the second display 132 can be pivotably mounted adjacent to an x-ray source 140 to be viewable and accessible for adjustment over a 360 degree area.

The displays 130, 132 can implement or control (e.g., by way of touch screens) functions such as rendering, storing, transmitting, modifying, and printing of an obtained image(s) and can cooperate with or include an integral or separate control panel, shown as control panel 150 in FIG. 2, to assist in implementing functions such as rendering, storing, transmitting, modifying, and printing an obtained image(s). One or more of displays 130, 132 can be separable from the apparatus frame 120. One or more of displays 130, 132 can act as display monitors for providing control messages and showing instruction entry.

For mobility, wheeled mobile radiographic apparatus 110 can have one or more wheels 112 and one or more handle grips 114, typically provided at waist-level, arm-level, or hand-level, that help to guide the mobile radiographic apparatus 110 to its intended location. A self-contained battery pack (e.g., rechargeable, not shown) can provide source power, which can reduce or eliminate the need for operation near a power outlet. Further, the self-contained battery pack can provide for motorized transport between sites.

For storage, the mobile radiographic apparatus 110 can include an area/holder for holding/storing one or more digital radiographic (DR) detectors or computed radiography cassettes. The area/holder can be a storage area 136 (e.g., disposed on frame 120) configured to removably retain at least one digital radiography (DR) detector. Storage area 136 can be configured to hold a plurality of detectors and can also be configured to hold one size or multiple sizes of DR detectors and/or batteries therein.

Mounted to frame 120 is a support member 138, a column that supports one or more x-ray sources 140, also called an x-ray tube, tube head, or generator that can be mounted to support member 138. In the embodiment shown in FIG. 2, the supporting column (e.g., member 138) can include a second section, a type of boom that extends outward a fixed/variable distance from a first section where the second section is configured to ride vertically up and down the first section to the desired height for obtaining the image. In addition, the support column is rotatably attached to moveable frame 120. In another embodiment, the tube head or x-ray source 140 can be rotatably coupled to support member 138. In another exemplary embodiment, an articulated member of the support column that bends at a joint mechanism can allow movement of the x-ray source 140 over a range of vertical and horizontal positions. Height settings for x-ray source 140 can range from low height for imaging of feet, ankles, knees and lower extremities to shoulder height and above for imaging the upper body anatomy of patients in various positions.

In the ICU environment, mobile radiographic apparatus 110 can be used to provide imaging capabilities at the patient's bedside, reducing or eliminating the need to transport critically ill patients to other locations for routine imaging. Mobile radiography apparatus 110 can provide conventional x-ray imaging, in which a single image is obtained from a single exposure at a single exposure energy. Alternately, mobile radiography apparatus 110 can provide more advanced imaging capabilities, including spectral imaging that uses the combined information from two exposures of the same subject, the two exposures taken at different energy levels, and generates image content with computationally enhanced information based on differences between results from the two exposures.

In a tomosynthesis mode, mobile radiographic apparatus 110 can take a rapid succession of images of the subject at a series of changing angles in order to reconstruct a tomosynthesis or “depth” image. In a motion imaging mode, mobile radiography apparatus 110 can take a succession of images of the subject, wherein the images can be rendered in sequence in order to depict movement dynamics, such as for a joint or other structural anatomy.

Considerable depth and range of information can be derived from image content, which can often show subtle changes in patient condition that may not be readily detectable from measurement instrument data alone. Thus, the capability for using image analysis can add significantly to the speed and accuracy of diagnosis and of prognosis generation for the patient.

### Utilizing Test Results and Vital Signs Measurements

In addition to image content, there can be a considerable body of other test data, as well as periodic vital sign measurement information that is gathered from the patient during each shift, typically by technicians or nursing personnel. This data is recorded, but may not be correlated with other patient data until some time after it is obtained. It can be of particular value to combine test and vital sign data with image analysis from modules 10. The image processing logic described above can utilize the most current patient test results, as well as patient test history, to support analysis of image content.

As described previously with reference to FIGS. 1A and 1B, test measurements and results can be incorporated into patient characterization profile 100, which stores the patient data in a format that can more readily be usable for supporting image analysis in modules 10 and for prognosis generation process 200.

Vital signs data can also be recorded and input into patient characterization profile 100 for use with the image content analysis in prognosis generation process 200.

### Utilizing Patient History

An embodiment of the present disclosure can access the complete patient history, including both medical and other data, in conjunction with diagnostic image content and test and vital signs data in generating a prognosis for the patient. Machine learning routines can be trained using aspects of patient history as well as medical imaging and measurement data to help identify trends, patterns, and information that can influence prognosis logic.

### Patient Characterization Profile

As shown in FIGS. 1A and 1B, patient characterization profile 100 can serve as a receptable for packaging patient data from imaging, test, vital signs, and patient history sources. Patient characterization profile 100 can serve to collect and organize all applicable medical data that has been obtained for the patient and can further include data and observations from previous patient history. According to an embodiment, patient characterization profile 100 provides a structuring of data in a format that is usable for a prognosis generation process 200. Patient characterization profile 100 can also store data generated from learned logic processing.

### Prognosis Generation

Prognosis generation process 200 can be executed using machine learning (learned logic), with neural network logic formed by analysis and processing of numerous cases used for training.

Machine learning techniques have been successfully adapted to tasks that relate to image classification and feature recognition. Embodiments of the present disclosure can utilize machine learning for further processing image content for the ICU patient, adding the value of data on test measurements, vital signs, and the overall patient history, as described hereinabove.

For generating an accurate prognosis in any individual case, an embodiment of the present invention can focus on particular image types, for example: chest x-rays, MMR, ultrasound, etc. Various parts of the anatomy can be of interest, including skull, ribcage, internal organs, etc.

The machine learning models used can employ any of a number of appropriate machine learning types. Machine learning, as used herein can include supervised learning, in which labeled input and output examples are provided and system logic executes continuously in order to adjust internal variables and cost functions that direct decision-making in the internal logic. Supervised learning can use any of a number of known techniques including regression logic, back propagation neural networks, random forests, decision trees, and other methodologies. Alternately, unsupervised learning methods can be adopted, such as using K-means clustering or a priori algorithms, for example.

Machine learning can alternately adopt various approaches such as semi-supervised learning or other suitable learning method. Reinforcement learning methods can be used, such as methods that employ a Q-learning algorithm or use temporal difference learning, or methods that are patterned on any other appropriate learning model.

Each portion of the machine learning application can implement any one or more of: a regression algorithm (e.g., ordinary least squares, stepwise regression, logistic regression, multivariate adaptive regression splines, locally estimated scatterplot smoothing, etc.), an instance-based method (e.g., k-nearest neighbor, learning vector quantization, self-organizing map, etc.), a regularization method (e.g., ridge regression, least absolute shrinkage and selection operator, elastic net, etc.), a decision tree learning method (e.g., classification and regression tree, iterative dichotomiser, chi-squared automatic interaction detection, decision stump, random forest, multivariate adaptive regression splines, or gradient boosting machine, for example), a Bayesian method (e.g., naïve Bayes, averaged one-dependence estimators, or Bayesian belief network), a kernel method (e.g., a support vector machine, a radial basis function, a linear discriminate analysis, etc.), a clustering method (e.g., k-means clustering, expectation maximization, etc.), an associated rule learning algorithm (e.g., an a priori algorithm or an Eclat algorithm), an artificial neural network model (e.g., a Perceptron method, a back-propagation method, a Hopfield network method, a self-organizing map method, or a learning vector quantization method), a deep learning algorithm (such as a restricted Boltzmann machine, a deep-belief network method, a convolution network method, a stacked auto-encoder method), a dimensionality reduction method (e.g., principal component analysis, partial lest squares regression, Sammon mapping, multidimensional scaling, projection pursuit), an ensemble method (such as boosting, bootstrapped aggregation, AdaBoost, stacked generalization, gradient boosting machine method, random forest method), and any suitable form of machine learning algorithm.

Modular design can be advantageous for learned logic applications. Each machine-learning processing portion of the system can additionally or alternatively follow the model for a probabilistic module, heuristic module, deterministic module, or any other suitable module that leverages any other suitable computational method, machine learning method, or combination thereof. Any suitable machine learning approach can be incorporated into the system as a learned logic module, as appropriate.

In order to execute various steps in the process flow shown herein, a processor configured to apply learned logic from machine learning can be trained to evaluate image quality and image content and features using deep learning methods. Deep learning learned logic (e.g., deep structured learning, hierarchical learning, or deep machine learning) models high-level abstractions in data. In deep learning, the input features required to train machine logic are not explicitly defined or engineered by the user, as is the case using more “shallow” or focused learning algorithms. The machine learning output can be highly abstract (for example, a judgement on image quality, assessment of the condition for the imaged patient anatomy) relative to the input. The input itself is typically a lengthy vector that lists pixel values.

Accuracy of prognosis using learned logic can be advanced by the use of DR and its more advanced image capture techniques, including bedside tomosynthesis, dual-energy or spectral imaging, and serial radiography, which can provide a measure of dynamic motion imaging. The combination of diagnostic image content with other types of patient metrics can help to alert, guide, and inform practitioners and care staff who are often pressed for time and working under considerable duress in the ICU environment.

Deep learning is a subset of machine learning that uses a set of algorithms to model high-level abstractions in data using a deep graph with multiple processing layers including linear and non-linear transformations. While many machine learning systems are seeded with initial features and/or network weights to be modified through learning and updating of the machine learning network, a deep learning network trains itself to identify “good” features for analysis. Using a multilayered architecture, machines employing deep learning techniques can often process raw data better than machines using conventional machine learning techniques, particularly where judgment and analysis/assessment normally reserved for the skilled practitioner/observer have normally been needed. Examining data for groups of highly con-elated values or distinctive themes is facilitated using different layers of evaluation or abstraction.

Deep learning in a neural network environment includes numerous interconnected nodes referred to as neurons. Input neurons, activated from an outside source, activate other neurons based on connections to those other neurons which are governed by the machine parameters. A neural network behaves in a certain manner based on its own parameters. Learning refines the machine parameters, and, by extension, the connections between neurons in the network, such that the neural network behaves in a desired manner.

A neural network provides deep learning by using multiple processing layers with structures adapted to provide multiple non-linear transformations, where the input data features are not engineered explicitly. In embodiments of the present disclosure, a deep neural network can process the input image data content by using multiple layers of feature extraction to identify features of the image content, such as for image quality measurement or for assessing patient condition. The machine logic training itself is typically run in unsupervised mode, learning the features to use and how to classify given an input sample (i.e., feature vector). Other deep learning, sparse auto-encoding models may alternately be trained and applied for one or more processes in the sequence.

### Reporting

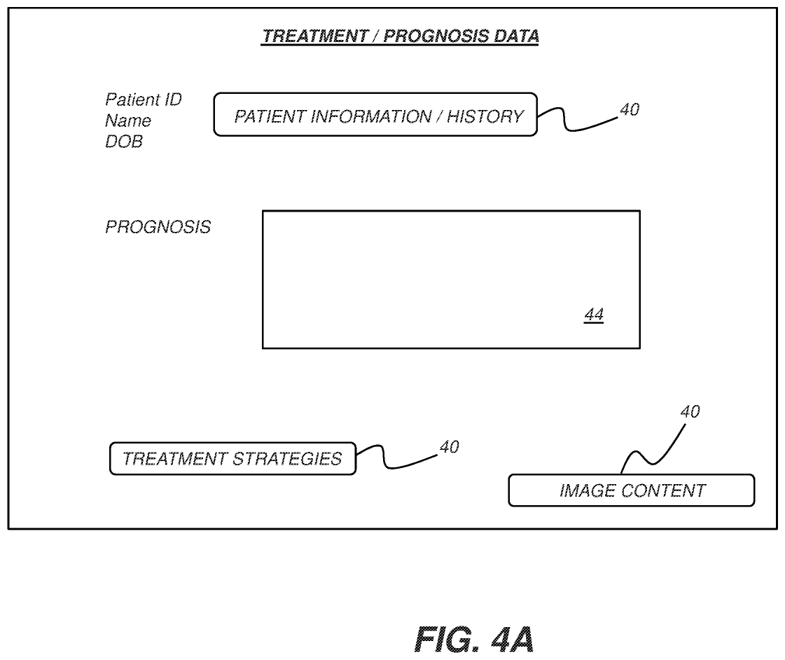

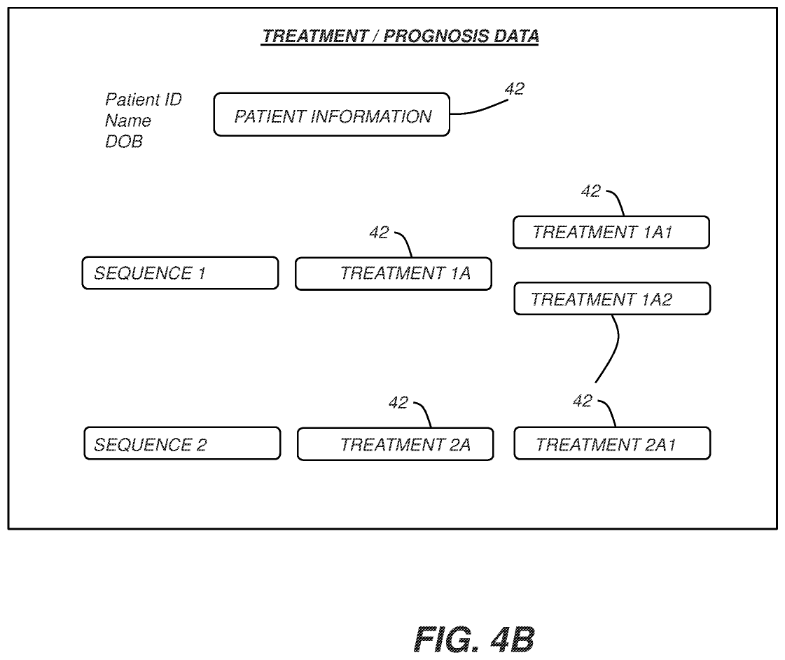

FIGS. 4A and 4B show exemplary interface display arrangements that can provide useful reporting of prognosis data and projected treatment strategies. The example of FIG. 4A shows an introductory screen for the ICU patient, listing identifying information and providing one or more controls 40 for selection by the user in order to view more information about the patient, such as patient or treatment history, for example, and, optionally, to view one or more treatment strategies determined by the system, based on the patient information. A preliminary prognosis statement 44 can be provided, with summary data generated by system logic.

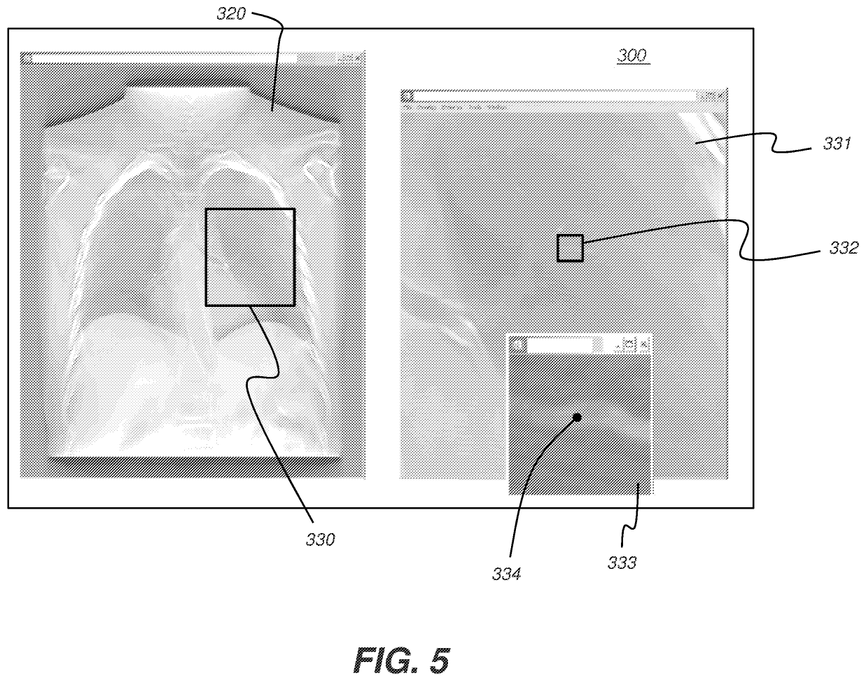

As shown in the example of FIG. 4A, a control 40 can appear when the learned logic has identified a location or region of interest (ROI) within diagnostic image content. Selection of this control 40 can display the image content, as shown for a tomosynthesis image in the example of FIG. 5.

By way of example, FIG. 5 shows a display screen 300 with a rendering of a tomosynthesis reconstruction 320 obtained using a set of x-ray projection images acquired within a narrow angular range for a phantom chest image, and identifying by highlighting, using an outline 330, a particular area of interest that has been detected using learned logic and which detected area of interest may not be visible to a human eye in the reconstruction 320. The enlarged image 331 of the area of interest is displayed and the area of interest is again highlighted by outline 332 for viewing and for display manipulation functions (zoom, pan, crop) by the viewer/practitioner. As one simple form of highlighting, screen 300 can display outlined areas 330, 332, containing a feature of interest. Other highlighting methods can accentuate features of interest using contrast or color differences, for example. Successive enlargement 331, 333, of the area of interest highlighted by outlines 330, 332, respectively, can help to direct the practitioner's attention to a malignancy or other irregular feature 334 that may not be visible in the tomosynthesis reconstruction 320, for example.

FIG. 4B shows an exemplary arrangement for a follow-on screen display generated by prognosis generation process 200. Controls 42 can link to other displayed data such as patient information or treatment information according to the generated prognosis. In the example shown, two alternative treatment sequences are outlined by the system in response to the generated prognosis data. Numerous other arrangements are possible; the number of alternative sequences and controls can vary according to results from the learned logic when applied for the particular patient. In addition, prognosis-related data can change based on viewer selection of a particular treatment strategy. This display can then help to guide the attending staff in executing a recommended treatment regimen appropriate for the patient.

The mobile radiography apparatus 110 can also maintain a record of patient exposure levels.

The process output, a prognostic outlook for the patient, can have any of a number of forms. According to an embodiment of the disclosure, the analysis processor can output probabilistic metrics or indicators. These indicators could include probability of mortality as a function of time and probability of disease progression as a function of time. Additional outputs may include recommendations or suggestions for changes in treatment regimen that can help to increase the probability (and timing) for a positive patient outcome. Changes in treatment regimen could include adjustments to respirator settings, flow rates for IV fluid, changes to antibiotic concentration, among others.

### Training Considerations

Time-related considerations can complicate the task of developing and executing a suitable training sequence for machine learning. There are few patterns for the scheduling of radiographic imaging for patients preceding or during a stay in the ICU; x-rays, for example, are typically obtained on an as-needed basis, with frequency of exam sessions and number of exposures widely varying over the ICU patient population. This means that image data presents a sparse-data problem for machine-learning, requiring corresponding techniques for developing decision logic. Sparse-data techniques of various types are known; these approaches generally draw inferences from the available information in order to provide sufficient data content for machine learning.

To take advantage of data available in the training set, image data used for machine training is indexed according to timing in embodiments of the present disclosure, so that where there is a sequence of images for the same patient in the training data, acquisition time for the examination session is provided as part of the training data.

Forming a suitable training set for input to a neural network or other machine-learning processor can be a formidable task, collecting patient data of various types, including image data, along with prognosis and outcome information. A modular approach can be more efficient than attempts to generate machine logic from thousands of patient cases.

As one example of modular approach benefits: in order to reduce the complexity and number of processing steps that might be required for handling raw image data, it can be useful to handle the image processing functions separately, leveraging existing solutions for image analysis and deriving the needed data from radiographic image content, then submitting imaging results to the patient characterization profile 100 (FIG. 1A). The patient characterization profile 100 can then be directed to the neural network logic. In this way, some types of existing image processing logic can be integrated into the workflow for image analysis in order to extract results suitable for subsequent prognosis development from the radiographic image content and provide this data to the neural network system.

The invention has been described in detail, and may have been described with particular reference to a suitable or presently preferred embodiment, but it will be understood that variations and modifications can be effected within the spirit and scope of the invention. The presently disclosed embodiments are therefore considered in all respects to be illustrative and not restrictive. The scope of the invention is indicated by the appended claims, and all changes that come within the meaning and range of equivalents thereof are intended to be embraced therein.

1. A computer implemented method for evaluating diagnostic images of a patient, the method comprising:
acquiring and storing the diagnostic images of the patient each obtained during different examination sessions;
evaluating the diagnostic images of the patient using trained machine learning logic to generate prognosis and treatment information for the patient based on a medical condition detected in the diagnostic images during the evaluation; and
generating and outputting the prognosis and treatment information.

2. The computer implemented method of claim 1, further comprising generating and outputting a schedule for patient treatment according to the generated prognosis and treatment information.

3. The computer implemented method of claim 2, wherein outputting the schedule further comprises displaying human readable instructions for one or more treatment sequences.

4. The computer implemented method of claim 3, further comprising reconstructing a tomosynthesis image from acquired diagnostic x-ray images of the patient.

5. The computer implemented method of claim 4, wherein acquiring the diagnostic x-ray images of the patient comprises obtaining a spectral radiography image of the patient using a wheeled mobile radiography apparatus.

6. The computer implemented method of claim 1, wherein acquiring the diagnostic images of the patient comprises acquiring a progressive series of diagnostic images over time.

7. The computer implemented method of claim 1, further comprising:
acquiring and recording vital signs measurements of the patient; and
evaluating the diagnostic images of the patient and the acquired vital signs measurements of the patient using trained machine learning logic to generate the prognosis and treatment information for the patient.

8. The computer implemented method of claim 7, further comprising acquiring and recording heart rate data, blood pressure data, blood oxygen level data, lung fluid level data, or a combination thereof.

9. The computer implemented method of claim 8, further comprising outputting a probability of disease progression and/or mortality of the patient as a function of time.

10. The computer implemented method of claim 9, further comprising outputting recommendations for changes in a treatment regimen for the patient to improve a probability or timing of the patient's probability of disease progression or mortality.

11. The computer implemented method of claim 10, further comprising outputting changes to respirator settings, changes to flow rates for IV fluid, changes to antibiotic concentration, or a combination thereof.
